(19) United States
(12) Patent Application Publication
(10) Pub. No.: US 2004/0170580 A1
(43) Pub. Date: Sep. 2, 2004

(54) Filtering suntan product

(76) Inventors: Rainer Schmidt, Clamart (FR); Marcelle Regnier, Paris (FR); Christine Duval, La Gerennes Colombes (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/474,411

(22) PCT Filed: Apr. 9, 2002
(86) PCT No.: PCT/FR02/01238

(30) Foreign Application Priority Data
Apr. 9, 2001 (FR) 01 04808

(51) Int. Cl. A61K 7/42

(52) U.S. Cl. 424/59

(57) ABSTRACT

The invention concerns a product comprising at least a UV radiation filtering agent and at least a compound stimulating melanin synthesis, a composition comprising at least said product and the use of said product in a composition or for preparing a composition designed to protect the skin against the harmful action of UV radiation, as well as a cosmetic skin treatment method.

The invention relates to a product comprising at least one ultraviolet radiation-screening agent and at least one compound stimulating melanin synthesis, to a composition comprising at least said product, to the use of said product in a composition or for preparing a composition intended to protect the skin against the harmful action of ultraviolet radiation, and also to a cosmetic skin treatment method.

Solar radiation is made up, inter alia, of ultraviolet radiation type A having wavelengths of between 320 nm and 400 nm (UV-A), ultraviolet radiation type B having wavelenghts of between 280 and 320 nm (UV-B) and ultraviolet radiation type C having wavelengths of between 200 and 280 nm (UV-C).

UV-B radiation is highly energetic and relatively non-penetrating, is poorly represented in sunlight and is dependent on climatic variations (cloudy weather, cloud cover, etc.) and its presence varies as a function of the time of day (notion of peak (zenith)).

UV-A radiation is less energetic than UV-B radiation but more penetrating, is present in a large amount in sunlight (a minimum of 100 times more UV-A radiation than UV-B radiation), is relatively independent of climatic variations and is present whatever the time of day.

UV-C radiation is highly energetic and relatively non-penetrating. It is stopped by the ozone layer and theoretically does not reach earth. However, it can potentially be responsible for nucleic acid damage.

It is known that solar radiation is responsible for beneficial effects on the skin, such as, for example, browning, but it is also capable of inducing damage thereto, in particular in the case of “sensitive” skin or of continually exposed skin.

In respect of the benefits, browning, commonly referred to as tanning, is an essential element of the skin's defense system. Specifically, in response to ultraviolet radiation, the melanocytes of the basal layer of the epidermis synthesize melanin which, once incorporated into the keratinocytes, constitutes a natural screen located at the surface of the skin, which screen absorbs the ultraviolet radiation. The melanin, in the form of particles, can also act as a UV-reflecting screen. The aim of this is to decrease the amount of ultraviolet radiation which crosses the layers of skin in order to prevent it reaching the deep layers and causing damage therein which is harmful to the skin.

It is, moreover, known just how important it is to look well and how a tanned skin is always the sign of good health; hence the craze for prolonged exposure to ultraviolet radiation in order to acquire a sufficient tan.

However, in respect of the harmful effects, it is known that excessive exposure of the skin to solar radiation, and to ultraviolet radiation in particular, can lead to a change in the elasticity of the skin, and in its content of certain compounds, and can thus promote acceleration of the natural process of skin aging. This accelerated or premature aging process due to ultraviolet radiation is generally referred to as photoaging or actinic aging or dermatoheliosis.

Weakly penetrating UV-B radiation reaches mainly the epidermis. The role of UV-B radiation has been clearly demonstrated in the induction of UV-induced skin cancers. Its main chromophore is in fact nucleic acids, in particular deoxyribonucleic acid, within which it induces damage and/or mutations (Eller M. S., 1995, in Photodamage. 26-56, Blackwell ed.).

UV-A radiation which crosses the epidermis and reaches the dermis is highly involved in photoaging of the skin: it participates, for example, in the appearance of solar elastosis. Moreover, it is known that photosensitizing reactions and photodermatoses such as polymorphic dermatitis are mediated mainly by UV radiation.

UVA radiation is also mutagenic (Stary A. Mutation Res. 1997; 398: 1-8) and photocarcinogenic (De Laat A., 1998, in Protection of the skin against ultraviolet radiations. 19-23, John Libbey Eurotext ed) and, along with UVB radiation, can contribute to the development of skin cancers.

With the development of tanning salons or the use of a UVA lamp at home, promoting massive and prolonged exposure to UVA radiation, it has been shown that UVA radiation modifies the melanocyte and causes the appearance of pigmentary blemishes (lentigo) and an increase in the risk of developing a melanoma (Runger TM Photodermatol. Photoimmunol. Photmed. 1999; 15-212-216).

The dilemma which exists of, on the one hand, wishing to expose oneself to ultraviolet radiation in order to have a tanned appearance and, on the other hand, the absolute need to protect oneself against this same radiation due to its harmful effects is therefore understandable.

To date, one of the solutions proposed is to provide compositions comprising a screening agent having a medium protection index allowing both a certain amount of protection and a certain amount of tanning. In fact, if the screening agent is present in a high amount, it will limit all the more the stimulation, by the ultraviolet radiation, of the melanin synthesis by the melanocytes. Conversely, a composition containing a low or medium amount of screening agent does not provide sufficient guarantees in terms of protection against the harmful effects of the ultraviolet radiation, nor does it provide sufficient satisfaction in terms of obtaining a tan.

Moreover, certain combinations for reinforcing tanning of the skin, with a sunscreen, have been proposed.

Compositions containing psoralen derivatives and a screening agent have thus been described in FR 2409751 or FR 2797585. However, to promote pigmentation, psoralen derivatives such as 5-methoxypsoralen require simultaneous irradiation of the skin with doses of UVA, which can damage the DNA; in addition, the use of such psoralen derivatives is not desirable since they may be involved in photosensitizing reactions.

Combinations of UV screening agents with tyrosine or xanthines have also been proposed, in particular in FR 2 624374 or FR 2607699; however, the activity of these preparations is not demonstrated and they also require notable irradiation of the skin, and often additional adjuvants.

CH 642357 describes compositions containing tyrosine derivatives, UV screening agents and chromophoric derivatives.

WO 91/07945 or EP 380335 proposes the use of xanthines to promote tanning, optionally combined with a sunscreen. However, the effectiveness of the xanthines as a tanning agent in humans is not satisfactory. WO 98/25584 claims anti-inflammatory and melanogenesis-activating compositions containing derivatives of αMSH and SOD, to which titanium oxide may be added. It is known that the derivatives of this hormone can have activities on various functions of the organism and, in addition, have a low pro-pigmenting capacity.

There remains therefore a need for a screening composition which would exhibit, firstly, a high screening capacity and, secondly, an ability to stimulate tanning in equally high proportions.

The applicant has now just shown that the combination of an ultraviolet radiation-screening agent and a compound stimulating melanin synthesis exhibits a surprising effect which leads to the production of a screening suntan product having high screening qualities and a high capacity for stimulating melanin synthesis.

A first subject of the invention is therefore a product made up of at least the combination of at least one ultraviolet radiation-screening agent and an agent stimulating melanin synthesis.

These compositions solve the problem of obtaining tanning and protection of the skin equivalent to that obtained after exposure to the sun, without subjecting it to the risks induced by irradiation with UV radiation, in particular UVA radiation.

A subject of the invention is also a composition comprising at least the combination of at least one ultraviolet radiation-screening agent and an agent stimulating melanin synthesis.

Preferably, the agent stimulating melanin synthesis is an agent which is not liable to cause systemic side effects on the organism, in particular hormonal effects, or phenomena of photosensitization.

The ultraviolet radiation-screening agent is preferably chosen from organic screening agents and/or inorganic screening agents.

As organic screening agents, mention may in particular be made of cinnamic derivatives, salicylic derivatives, camphor derivatives, triazine derivatives, benzophenone derivatives, dibenzoylmethane derivatives, β,β-diphenylacrylate derivatives, p-aminobenzoic acid derivatives, the screening polymers and screening silicones described in application WO-93/04665 or else the organic screening agents described in patent application EP-A 0 487 404.

As inorganic screening agents, mention may in particular be made of pigments or else of nanopigments (mean size of the primary particles: generally between 5 nm and 100 nm, preferably between 10 and 50 nm) of metal oxides which may or may not be coated, such as, for example, nanopigments of titanium oxide (amorphous or crystallized in rutile and/or anatase form), iron oxide, zinc oxide, zirconium oxide or cerium oxide, which are all photoprotective agents well known per se, which act by physical blocking (reflection and/or scattering) of the UV radiation. Conventional coating agents are, moreover, alumina and/or aluminum stearate. Such coated or uncoated metal oxide nanopigments are in particular described in patent applications EP-A-0 518 772 and EP-A-0 518 773.

As examples of sunscreens which are active in the UV-A and/or UV-B range, mention may be made of:

p-aminobenzoic acid,

oxyethylenated (25 mol) p-aminobenzoate,

2-ethylhexyl p-dimethylaminobenzoate,

N-oxypropylenated ethyl p-aminobenzoate,

glyceryl p-aminobenzoate,

homomenthyl salicylate,

2-ethylhexyl salicylate,

triethanolamine salicylate,

4-isopropylbenzyl salicylate,

4-tert-butyl-4′-methoxydibenzoylmethane,

4-isopropyldibenzoylmethane,

menthyl anthranilate,

2-ethylhexyl 2-cyano-3,3′-diphenylacrylate

ethyl 2-cyano-3,3′-diphenylacrylate,

2-phenylbenzimidazole-5-sulfonic acid and salts thereof,

3-(4′-trimethylammonium)benzylidenebornan-2-one methyl sulfate,

2-hydroxy-4-methoxybenzophenone,

2-hydroxy-4-methoxybenzophenone 5-sulfonate,

2,4-dihydroxybenzophenone,

2,2′,4,4′-tetrahydroxybenzophenone,

2,2′-dihydroxy-4,4′-dimethoxybenzophenone,

2-hydroxy-4-n-octoxybenzophenone,

2-hydroxy-4-methoxy-4′-methylbenzophenone,

α-(2-oxoborn-3-ylidene)tolyl-4-sulfonic acid and salts thereof,

3-(4′-sulfo)benzylidenebornan-2-one and salts thereof,

3-(4′-methylbenzylidene)-d,l-camphor,

3-benzylidene-d,l-camphor,

benzene-1,4-bis(3-methylidene-10-camphorsulfonic) acid and salts thereof,

urocanic acid,

2,4,6-tris[p-(2′-ethylhexyl-1′-oxycarbonyl)anilino]-1,3,5-triazine,

2-[(p-(tert-butylamido)anilino]-4,6-bis[(p-(2′-ethylhexyl-1′-oxycarbonyl)anilino]-1,3,5-triazine,

2,4-bis{[4-2-ethylhexyloxy)]-2-hydroxy]phenyl}-6-(4-methoxyphenyl)-1,3,5-triazine;

N-(2 and 4)-[(2-oxoborn-3-ylidene)methyl]benzyl-acrylamide polymer,

4,4-bisbenzimidazolylphenylene-3,3′,5,5′-tetrasulfonic acid and salts thereof,

2,2′-methylenebis[6-(2H-benzotriazol-2-yl)-4-(1,1,3,3-tetramethylbutyl)phenol],

polyorganosiloxanes containing a malonate function.

The amount of compound acknowledged to be an ultraviolet radiation-screening agent, contained in the composition of the invention, depends of course on the desired effect and may therefore vary to a large extent.

Use will preferably be made of agents capable of substantially screening UVA radiation. According to an advantageous embodiment of the invention, the IP UVA (UVA protection index) determined according to the method of the JCIA (Japanese Cosmetic Industry Association, Measurement standards for UVA efficacy. Tokyo, Japan: 1995) will be from 5 to 20, preferably from 8 to 15.

Advantageously, the screening preparation will have a sun protection factor or SPF, determined according to the Colipa method (The European cosmetic toiletry and perfumery association (Colipa). Sun protection factor method. Report 94/289. Brussels, Belgium, 1994), at least equal to 10 and less than or equal to 60, preferably from 15 to 30.

The screening preparation will thus contain an amount of UVB- and UVA-screening agents so as to combine a high SPF with an IP UVA which is also high.

Screening agents which are particularly suitable for implementing the invention will be chosen from the group comprising 2-ethylhexyl salicylate, 4-tert-butyl-4′-methoxydibenzoylmethane, 2-hydroxy-4-methoxybenzophenone-5-sulfonate, benzene-1,4-bis(3-methylidene-10-camphorsulfonic) acid and salts thereof, and the mixtures of these compounds.

To give an order of magnitude, the amount of ultraviolet radiation-screening agents contained in the composition is in an amount representing from 0.1% to 25% of the total weight of the composition, in particular from 6 to 25%; advantageously, it will be at least 8%, and preferably less than or equal to 15%, of the total weight of the composition; according to another advantageous embodiment of the invention, the screening agents are present in an amount representing from 0.5 to 10% of the total weight of the composition.

The agent(s) stimulating melanin synthesis can be chosen from:

analogs of substrates for tyrosinase, a key enzyme in melanogenesis, such as tyrosine, L-dopa or L-dihydroxyphenylalanine,

activators of tyrosinase activity or expression, such as forskolin, xanthine bases (theophylline, caffeine), pro-opiomelanocorticotropic peptides (ACTH, alpha-MSH or other MC1 receptor agonists), diacylglycerols, aliphatic or cyclic diols, psoralens, prostaglandins and analogs, activators of NO/cGMP-dependent protein kinase G,

activators of transfer of melanosomes to keratinocytes, such as serine proteases, or of PAR-2 receptor agonists.

According to the invention, use is preferably made of an extract obtained from a plant, in particular from Burnet (Sanguisorba officinalis) or else an extract of at least one plant of the Chrysanthemum genus, particularly of the species Chrysanthemum sinensis.

In fact, surprisingly, the applicant has demonstrated that such extracts induce a notable stimulation of melanogenesis and therefore of tanning, even in the absence of notable irradiation with UVA radiation. This unexpected effect is much greater than that obtained with other propigmenting agents. The extract of at least one plant of the Chrysanthemum genus can be any extract prepared from any plant material derived from a plant of the Chrysanthemum genus.

The composition can contain at least one extract of at least one plant of the Chrysanthemum genus, obtained from material derived from a plant grown in vivo or derived from culturing in vitro.

“Culturing in vitro” is intended to mean any culture of the conventional type, i.e. in soil in the open air or under glass, or else out of soil.

According to the invention, it is thus possible to use, for example, an extract of various parts of the plant, leaves, flowers, stems, roots, undifferentiated cells, alone or as a mixture, whether the plant is grown in vivo or in vitro.

The selection pressure imposed by the physicochemical conditions during the growth of the plant cells in vitro makes it possible to obtain a standardized plant material available throughout the year, unlike plants grown in vivo.

“Culturing in vitro” is intended to mean all the techniques known to those skilled in the art which make it possible to artificially obtain a plant or a part of a plant.

Use is preferably made of an extract obtained from plant material grown in vivo, and even more preferably an extract obtained from leaves of plants of the Chrysanthemum genus grown in vivo.

The expression “extract of at least one plant of the Chrysanthemum genus” is intended to mean both a crude mixture of parts of the plant roughly reduced to pieces and of the extraction solvent, and preparations developed from the active principles solubilized during the extraction. Use may in particular be made of extracts prepared according to the techniques described in application FR 2768343, which is incorporated herein by way of reference. An extract which can particularly be used according to the invention is prepared by fine grinding of parts of the plant, followed by soaking in the extraction solvent, said solvent preferably being a hydrophilic solvent, and, finally, by filtration. Quite obviously, the extract according to the invention may be each one of the extracts thus obtained, used alone or as a mixture with one or more of the other extracts.

Any extraction method known to those skilled in the art can be used according to the invention.

Mention may in particular be made of aqueous extracts, alcoholic, in particular ethanolic, extracts, and aqueous-alcoholic extracts.

Whatever the form of the extract whose use is desired, the techniques used to obtain it are those generally described in the prior art and well known to those skilled in the art.

Use may also be made of an extract prepared by the method described in French patent application No. 95-02379.

Thus, in a first step, the plant material is ground in an aqueous solution under cold conditions; in a second step, the particles in suspension are removed from the aqueous solution derived from the first step; and in a third step, the aqueous solution derived from the second step is sterilized. This aqueous solution corresponds to the extract.

Furthermore, the first step can advantageously be replaced with a procedure consisting of simple freezing of the plant tissues (for example at −20° C.), followed by an aqueous extraction repeating the second and third steps described above.

The extract which can be used according to the invention may undergo successive fractionation steps for the purpose of concentrating the active principles. Advantageously, this fractionation will be performed by liquid/liquid extraction; use will preferably be made of the fractions extracted with an aqueous-alcoholic mixture, eliminating the top fractions.

Whatever the method of preparation used according to the invention, subsequent steps aimed at promoting conservation and/or stabilization can be added without, however, modifying the very nature of the extract. Thus, for example, the extract obtained can be lyophilized by conventional lyophilization methods. A powder is thus obtained, which can be used directly or else mixed in a suitable solvent before use.

According to the invention an aqueous extract is preferably used.

As extract of Burnet, use is preferably made, according to the invention, of a dry extract of root and of rhizome of Sanguisorba officinalis, which can in particular be obtained in the form of a powder from Maruzen Pharmaceuticals Co., Ltd. (Burnet Extract Powder).

The amount of compound stimulating melanin synthesis contained in the composition of the invention depends, of course, on the desired effect and can therefore vary to a large extent.

To give an order of magnitude, the compound stimulating melanin synthesis contained in the composition is in an amount representing from 0.01% to 15% of the total weight of the composition, and preferably in an amount representing from 0.5 to 5%, in particular from 1 to 5%, of the total weight of the composition.

The compositions according to the invention can also contain artificial tanning and/or browning agents for the skin (self-tanning agents), such as, for example, dihydroxyacetone (DHA).

The compositions of the invention can also comprise conventional cosmetic adjuvants, in particular chosen from fatty substances, organic solvents, thickeners, softeners, antioxidants, opacifiers, stabilizers, emollients, hydroxy acids, antifoams, moisturizers, vitamins, fragrances, preserving agents, surfactants, fillers, sequestering agents, propellants, basifying or acidifying agents, colorants, or any other ingredient usually used in cosmetics, in particular for the production of antisun compositions in the form of emulsions.

The fatty substances may consist of an oil or a wax or mixtures thereof, and they also comprise fatty acids, fatty alcohols and fatty acid esters. The oils may be chosen from animal, plant, mineral or synthetic oils and in particular from liquid petroleum jelly, liquid paraffin, volatile or non-volatile silicone oils, isoparaffins, fluoro oils and perfluoro oils. Similarly, the waxes may be chosen from animal waxes, fossil waxes, plant waxes, mineral waxes and synthetic waxes that are known per se.

Among the organic solvents, mention may be made of lower alcohols and polyols.

The thickeners may in particular be chosen from crosslinked homopolymers of acrylic acid, and guar gums and celluloses which may or may not be modified, such as hydroxypropylated guar gum, methylhydroxyethyl-cellulose, hydroxypropylmethylcellulose or else hydroxyethylcellulose.

Of course, those skilled in the art will take care to select this or these optional additional compounds and/or the amounts thereof such that the advantageous properties, in particular the level of photoprotection, intrinsically linked to the binary combination in accordance with the invention are not, or not substantially, adversely affected by the addition(s) envisioned.

The compositions of the invention may be prepared according to techniques that are well known to those skilled in the art, in particular those intended for preparing emulsions of the oil-in-water or water-in-oil type, or else anhydrous compositions.

This composition may in particular be in the form of a simple or complex emulsion (O/W, W/O, O/W/O or W/O/W emulsion) such as a cream, a milk, a gel or a cream-gel, of a powder, of a solid composition or of flexible pastes, and may optionally be packaged as an aerosol and be in the form of a foam or a spray.

When it is an emulsion, the aqueous phase of this emulsion may comprise a nonionic vesicular dispersion prepared according to known processes (Bangham, Standish and Watkins. J. Mol. Biol. 13, 238 (1965), FR 2315991 and FR 2416008).

The cosmetic composition of the invention may be used as a composition for protecting the human epidermis or the hair against ultraviolet rays, as an antisun composition or as a make-up product.

When the cosmetic composition according to the invention is used for protecting the human epidermis against UV rays, or as an antisun composition, it may be in the form of a suspension or a dispersion in solvents or fatty substances, in the form of a nonionic vesicular dispersion or else in the form of an emulsion, preferably of oil-in-water type, such as a cream or a milk, or in the form of an ointment, a gel, a cream-gel, a stick, flexible pastes, an aerosol foam or a spray.

When the cosmetic composition according to the invention is used for protecting the hair, it may be in the form of a shampoo, a lotion, a gel, an emulsion or a nonionic vesicular dispersion and may constitute, for example, a rinse-out composition, to be applied before or after shampooing, before or after dyeing or bleaching, before, during or after permanent-waving or straightening the hair, a styling or treating lotion or gel, a blow-drying or hairsetting lotion or gel, or a composition for permanent-waving, straightening, dyeing or bleaching the hair.

When the composition is used as a make-up product for the eyelashes, the eyebrows or the skin, such as an epidermal treatment cream, a foundation, a tube of lipstick, an eye shadow, a face powder, a mascara or an eyeliner, it may be in solid or pasty, anhydrous or aqueous form, for instance oil-in-water or water-in-oil emulsions, nonionic vesicular dispersions or suspensions.

As a guide, for the antisun formulations in accordance with the invention which contain a vehicle of oil-in-water emulsion type, the aqueous phase (comprising in particular hydrophilic screening agents) generally represents from 50 to 95% by weight, preferably from 70 to 90% by weight, relative to the total formulation, the oily phase (comprising in particular lipophilic screening agents) represents from 5 to 50% by weight, preferably from 10 to 30% by weight, relative to the total formulation, and the (co)emulsifier(s) represent(s) from 0.5 to 20% by weight, preferably from 2 to 10% by weight, relative to the total formulation.

A subject of the invention is also the use of the combination of at least one ultraviolet radiation-screening agent and one or more agents stimulating melanin synthesis, in a screening suntan composition or for preparing a screening suntan composition.

A subject of the invention is also a cosmetic skin treatment method intended to protect it against the effects of UV rays while at the same time conferring on it a natural tan, consisting in applying to the skin an effective amount of a product as defined above or of a cosmetic composition comprising it.

The following examples and compositions illustrate the invention without in any way limiting it. In the compositions, the proportions indicated are percentages by weight.

### EXAMPLE 1

### Preparation of an Extract of Chrysanthemum sinensis

Leaves from Chrysanthemum sinensis plants grown in a greenhouse are removed and dried for 48 hours in a ventilated incubator at a temperature of 45° C.

The dried leaves are then reduced to powder by grinding in a knife grinder of the Culatti type.

The powder obtained is sieved through a screen in which the holes are 1 mm in diameter.

This sieved powder is used to prepare the extract.

Protocol 1:

The powder is mixed with an aqueous extraction solvent consisting of DMEM/F12, 3:1, cell culture medium sold by the company Life Technologies, at a concentration of 5 grams of dry powder per 100 ml of solvent. The mixture is stirred for 4 hours at ambient temperature. The mixture is then centrifuged at 1 000 rpm for 8 minutes and the supernatant is removed and subjected to two identical centrifugation/sampling cycles.

The final supernatant taken is filtered through a 0.22 μm filter of the Millipore type under aseptic conditions in order to be sterilized, and conserved at a temperature of 4° C. until use.

Protocol 2:

The powder is mixed with sterile demineralized water having a pH of 6.5 at a concentration of 2.5 grams of dry powder per 100 ml of water. The mixture is stirred for 30 minutes at ambient temperature. The mixture is then filtered through GFD membranes sold by the company Whatmann and having a porosity of 0.7 μl. The filtrate obtained is then filtered through a 0.22 μm filter of the Nalgene type under aseptic conditions in order to be sterilized, and conserved at a temperature of 4° C. until use.

Protocol 3:

The previous protocol is carried out, replacing the water with an aqueous extraction solvent consisting of DMEM/F12, 3:1, cell culture medium sold by the company Life Technologies.

Protocol 4:

The extract obtained in protocol 2 is lyophilized at 30° C. after freezing at −20° C. The powder obtained is used directly.

### EXAMPLE 2

### Measurement of the photoprotective and propigmenting effect of the combination of Mexoryl SX® (3,3′-(1,[lacuna]′phenylenedimethylidyne)bis(7,7-dimethyl-2-oxobicyclo[2,2,1]heptane-1-methanesulfonic acid) with either an extract of Burnet or an extract of Chrysanthemum sinensis obtained by protocol 2 of example 1

The protoprotective and propigmenting effect of the combination was tested on normal human keratinocyte/melanocyte cocultures according to the method described in patent FR 95/06491, by evaluating the UV-induced melanogenesis. The rate of melanin synthesis is evaluated through the incorporation of C¹⁴-labeled thiouracil. It is related to the amount of protein.

Normal human keratinocytes (NHKs) and normal human melanocytes (NHMs) are cultured from foreskin. The two types of cell are amplified and stored frozen. Eight days before the test, each of the cell types is again placed in culture. The keratinocytes are cultured according to the method described by Rheinwald and Green (Cell (1975) 6, 331-334) on feeder cells (3T3 fibroblasts) in Green 7F growth medium (DMEM/F12, 3:1, 10% fetal calf serum (Gibco), 0.18 mM adenine, 10 ng/ml epidermal growth factor EGF, 0.4 μg/ml hydrocortisone, 5 μg/ml insulin, 10 μM isoproterenol, 5 μg/ml transferrin and 2 nM triiodothyronine). The melanocytes are amplified in M2 medium (Olsson, M. J. et al., Lancet (1992) 340, 981). The media are changed every two days.

3 days before the test, 250 000 normal human keratinocytes and 80 000 normal human melanocytes are mixed and seeded per well of 24-well plates (Costar type) and cultured for three days in the keratinocyte growth medium (Green 7F) without phenol red. During the following four days, the culture medium is replaced daily with UV medium (DMEM/F12, 3:1, without phenol red, 2% defined calf serum (HyClone), 10 ng/ml EGF, 10 ng/ml fibroblast growth factor type β (βFGF)).

The UV-induction of pigmentation is carried out by solar simulated radiation (SSR) using a 1 000 watt Oriel Xenon solar simulator comprising a 280-400 nm dichroic filter (Oriel, model 81035) and a Schott WG 320 filter having a wavelength cut-off of 311 nm (corresponding to a thickness of approximately 1.5 mm).

The cell cultures are irradiated once a day for 4 days in a phosphate buffer solution (PBS).

The propigmenting molecules are brought into contact with the cells in the PBS during the irradiation and added to the culture medium (UV medium) after irradiation.

The extract of Burnet is tested at 0.005%. It is prepared in a stock solution at 0.5% in a solvent (dimethyl sulfoxide, DMSO), which is added at a 1/1 000 dilution to the PBS and to the UV medium. The final concentration of the solvent in the culture medium is therefore 1/1 000.

The extract of Chrysanthemum sinensis obtained according to protocol Nos. 2+4 is tested at 1%.

The tyrosine, which serves as a positive control, is tested at 500 μM.

The Mexoryl SX® is tested at 8%.

| INGREDIENTS | AMOUNT |
|---|---|
| WATER | 60 |
| TEREPHTHALYLIDENE | 8 |
| DICAMPHOR SULFONIC ACID | |
| PROPYLENE GLYCOL | 8 |
| GLYCEROL | 7 |
| SILICONE OIL | 8 |
| C12-C15 ALKYL BENZOATE | 2 |
| STEARYL ALCOHOL | 1.5 |
| PVP/EICOSENE COPOLYMER | 1 |
| SODIUM STEARYL GLUTAMATE | 1 |
| STEARIC ACID | 1.5 |
| PEG-100 STEARATE | 0.75 |
| GLYCERYL STEARATE | 0.75 |
| CARBOMER | 0.3 |
| HYDROXYPROPYLMETHYLCELLULOSE | 0.1 |
| TRIETHANOLAMINE | QS pH 7 |
| PRESERVING AGENT | QS |
| FRAGRANCE | QS |

After each exposure to UV radiation, the formulations containing the sunscreen are applied, in the proportion of 1.4 mg/cm², to a quartz slide on which there is a Transpore® strip, according to a method derived from that used to determine sun protection indices in vitro, described by Diffey and Robson (J Soc Cosmet Chemists (1989) 300, 230-235). The assembly is placed above the cocultures for the duration of the irradiation.

After irradiation, the cells are incubated in the UV medium containing 1 μCi/ml of C¹⁴-labeled thiouracil.

Measurement of Melanogenesis:

The following controls are realized:

control culture: no product tested

solvent control culture: the solvent of the products tested is added

positive control for stimulation of melanogenesis: 500 μM tyrosine;

24 hours after the final UV irradiation, the cells are rinsed in phosphate buffer. The proteins are precipitated with 5% trichloroacetic acid (TCA) and washed in order to remove the free radioactivity. The cells are lyzed overnight at 40° C. using a solution of proteinase K at 100 μg/ml in Tris HCl-triton-EDTA buffer.

5 μl of total extract are removed and transferred into a 96-well plate (Wallac) for the protein assay, which is carried out with the MicroBCA *Protein Assay Reagent kit (Pierce).

The rest of the extract, namely 950 μl, is filtered through a DEAE Filtermat filter. After rinsing, the filter covered with the “Meltilex” solid scintillant is transferred into a plate. The radioactivity is counted using a Wallac counter. The results are expressed as percentage of the control, or of the solvent control if the product is solubilized in a solvent other than water, according to the formulae:

$$\frac{( 14 CP \text{/amount protein} P ) - ( 14 CT \text{/amount protein} T )}{( 14 CT \text{/amount protein} T )} \times 100 and$$

$$\frac{( 14 CP \text{/mg protein} P ) - ( 14 CS \text{/mg protein} S )}{( 14 CS \text{/amount protein} S )} \times 100$$

in which:

¹⁴CP is the mean disintegration per minute (dpm) of ¹⁴C-thiouracil over three similar wells treated with a product (P);

amount protein P is the mean of the amounts of proteins in the corresponding wells (in milligrams),

¹⁴CT is the mean dpm of ¹⁴C-thiouracil over 3 similar control wells (T);

amount protein T is the mean of the amounts of proteins in the corresponding wells (in milligrams),

¹⁴CS is the mean dpm of ¹⁴C-thiouracil over 3 similar solvent control wells (S);

amount protein S is the mean of the amounts of proteins in the corresponding wells (in milligrams).

Results: % variation in melanin synthesis compared to the control (cells not treated with the product and not irradiated)

| Products | A |
|---|---|
| Irradiation alone (SSR) | 222 |
| Irradiation alone + solvent | 251 |
| 500 μM tyrosine | 40 |
| 0.005% extract of Burnet (solvent) | 110 |
| 1% extract of Chrysanthemum sinensis | 500 |
| SSR + 8% Mexoryl SX ® | 15 |
| SSR + solvent + 8% Mexoryl SX ® | 36 |
| SSR + 500 μM tyrosine | 382 |
| SSR + 0.005% extract of Burnet (solvent) | 466 |
| SSR + 1% extract of Chrysanthemum sinensis | 588 |
| SSR + 500 μM tyrosine + 8% Mexoryl SX ® | 74 |
| SSR + 0.005% extract of Burnet + 8% Mexoryl SX ® | 460 |
| (solvent) | |
| SSR + 1% extract of Chrysanthemum sinensis + 8% | 428 |
| Mexoryl SX ® | |

A = ¹⁴CthioU/amount protein (%/control)

The variations in melanin synthesis measured under the various conditions produce the following classification:

SSR+MSX<SSR+MSX+propigmenting agent< or =SSR+propigmenting agent.

### CONCLUSIONS

These results show that, in combination with a screening agent at high concentration, a propigmenting product allows tanning (melanin synthesis) to be restored.

### EXAMPLE 3

### Screening Suntan Composition to be Applied to the Skin Containing an Extract of Burnet at 1%

| | % |
|---|---|
| Water | 60 |
| Terephthalylidene dicamphor sulfonic acid | 8 |
| Propylene glycol | 8 |
| Glycerol | 7 |
| Silicone oil | 8 |
| Alcohol 12-15 alkyl benzoate | 2 |
| Stearyl | 1.5 |
| PVP/eicosene copolymer | 1 |
| Sodium stearoyl glutamate | 1 |
| Stearic acid | 1.5 |
| PEG-100 stearate | 0.75 |
| Glyceryl stearate | 0.75 |
| Carbomer | 0.3 |
| Hydroxypropylmethylcellulose | 0.1 |
| Triethanolamine | qs pH 7 |
| Preserving agent | qs |
| Fragrance | qs |

A dry extract of Sanguisorba officinalis root and rhizome, which can in particular be obtained in the form of a powder from Maruzen Pharmaceuticals Co., Ltd. (Burnet Extract Powder), is added to this composition such that the concentration of the dry extract in the finished product is 1%.

### EXAMPLE 4

### Screening Suntan Composition to be Applied to the Skin Containing Chrysanthemum sinensis at 1%

| | % |
|---|---|
| Water | 60 |
| Terephthalylidene dicamphor sulfonic acid | 8 |
| Propylene glycol | 8 |
| Glycerol | 7 |
| Silicone oil | 8 |
| Alcohol 12-15 alkyl benzoate | 2 |
| Stearyl | 1.5 |
| PVP/eicosene copolymer | 1 |
| Sodium stearoyl glutamate | 1 |
| Stearic acid | 1.5 |
| PEG-100 stearate | 0.75 |
| Glyceryl stearate | 0.75 |
| Carbomer | 0.3 |
| Hydroxypropylmethylcellulose | 0.1 |
| Triethanolamine | qs pH 7 |
| Preserving agent | qs |
| Fragrance | qs |

An extract of Chrysanthemum sinensis, obtained according to FR 2768343, is added to this composition such that the concentration of the extract in the finished product is 1%.

1. A product made of at least the combination of at least one ultraviolet radiation-screening agent and at least one agent stimulating melanin synthesis.

2. The product as claimed in claim 1, characterized in that the ultraviolet radiation-screening agent is chosen from organic screening agents and/or inorganic screening agents.

3. The product as claimed in either one of claims 1 and 2, characterized in that the ultraviolet radiation-screening agent is an organic screening agent chosen from cinnamic derivatives, salicylic derivatives, camphor derivatives, triazine derivatives, benzophenone derivatives, dibenzoylmethane derivatives, β,β-diphenylacrylate derivatives, p-aminobenzoic acid derivatives, screening polymers and screening silicones.

4. The product as claimed in either one of claims 1 and 2, characterized in that the ultraviolet radiation-screening agent is an inorganic screening agent chosen from pigments or nanopigments of metal oxides which may or may not be coated, such as, for example, nanopigments of titanium oxide (amorphous or crystallized in rutile and/or anatase form), iron oxide, zinc oxide, zirconium oxide or cerium oxide.

5. The product as claimed in any one of claims 1 to 4, characterized in that the ultraviolet radiation-screening agent is a sunscreen which is active in the UV-A and/or UV-B range.

6. The product as claimed in any one of claims 1 to 5, characterized in that the ultraviolet radiation-screening agent is chosen from p-aminobenzoic acid,
oxyethylenated (25 mol) p-aminobenzoate,
2-ethylhexyl p-dimethylaminobenzoate,
N-oxypropylenated ethyl p-aminobenzoate,
glyceryl p-aminobenzoate,
homomenthyl salicylate,
2-ethylhexyl salicylate,
triethanolamine salicylate,
4-isopropylbenzyl salicylate,
4-tert-butyl-4′-methoxydibenzoylmethane,
4-isopropyldibenzoylmethane,
menthyl anthranilate,
2-ethylhexyl 2-cyano-3,3′-diphenylacrylate
ethyl 2-cyano-3,3′-diphenylacrylate,
2-phenylbenzimidazole-5-sulfonic acid and salts thereof,
3-(4′-trimethylammonium)benzylidenebornan-2-one methyl sulfate,
2-hydroxy-4-methoxybenzophenone,
2-hydroxy-4-methoxybenzophenone 5-sulfonate,
2,4-dihydroxybenzophenone,
2,2′,4,4′-tetrahydroxybenzophenone,
2,2′-dihydroxy-4,4′-dimethoxybenzophenone,
2-hydroxy-4-n-octoxybenzophenone,
2-hydroxy-4-methoxy-4′-methylbenzophenone,
α-(2-oxoborn-3-ylidene)tolyl-4-sulfonic acid and salts thereof,
3-(4′-sulfo)benzylidenebornan-2-one and salts thereof,
3-(4′-methylbenzylidene)-d,l-camphor,
3-benzylidene-d,l-camphor,
benzene-1,4-bis(3-methylidene-10-camphorsulfonic) acid and salts thereof,
urocanic acid,
2,4,6-tris[p-(2′-ethylhexyl-1′-oxycarbonyl)anilino]-1,3,5-triazine,
2-[(p-(tert-butylamido)anilino]-4,6-bis[(p-(2′-ethylhexyl-1′-oxycarbonyl)anilino]-1,3,5-triazine,
2,4-bis{[4-2-ethylhexyloxy)]-2-hydroxy]phenyl}-6-(4-methoxyphenyl)-1,3,5-triazine;
N-(2 and 4)-[(2-oxoborn-3-ylidene)methyl]benzyl-acrylamide polymer,
4,4-bisbenzimidazolylphenylene-3,31,5,5′-tetrasulfonic acid and salts thereof,
2,2′-methylenebis[6-(2H-benzotriazol-2-yl)-4-(1,1,3,3-tetramethylbutyl)phenol],
polyorganosiloxanes containing a malonate function.

7. The product as claimed in any one of claims 1 to 6, characterized in that the ultraviolet radiation-screening agent is in an amount representing from 0.1% to 25% of the total weight of the composition.

8. The product as claimed in any one of claims 1 to 7, characterized in that the ultraviolet radiation-screening agent is in an amount representing from 0.5% to 10% of the total weight of the composition.

9. The product as claimed in any one of claims 1 to 8, characterized in that the agent stimulating melanin synthesis is chosen from analogs of substrates for tyrosinase, such as tyrosine, L-dopa or L-dihydroxyphenylalanine, activators of tyrosinase activity or expression, such as forskolin, xanthine bases (theophylline, caffeine), pro-opiomelanocortico-tropic peptides (ACTH, alpha-MSH or other MCl receptor agonists), diacylglycerols, aliphatic or cyclic diols, psoralens, prostaglandins and analogs, activators of NO/cGMP-dependent protein kinase G, or activators of transfer of melanosomes to keratinocytes, such as serine proteases, or of PAR-2 receptor agonists.

10. The product as claimed in any one of claims 1 to 9, characterized in that the agent stimulating melanin synthesis is an extract of Burnet (Sanguisorba officinalis) or else an extract of at least one plant of the Chrysanthemum genus.

11. The product as claimed in any one of claims 1 to 10, characterized in that the agent stimulating melanin synthesis is an extract of at least one plant of the species Chrysanthemum sinensis.

12. The product as claimed in any one of claims 1 to 11, characterized in that the agent stimulating melanin synthesis is in an amount representing from 0.01% to 15% of the total weight of the composition.

13. The product as claimed in any one of claims 1 to 12, characterized in that the agent stimulating melanin synthesis is in an amount representing from 1% to 5% of the total weight of the composition.

14. A composition comprising at least one product as defined in any one of claims 1 to 13.

15. The use, in a composition or for preparing a composition, of at least one product as defined in any one of claims 1 to 13.

16. A cosmetic skin treatment method intended to protect it against the effect of UV rays while at the same time conferring on it a natural tan, consisting in applying to the skin an effective amount of a product or cosmetic composition as claimed in one of claims 1 to 15.
